(19) United States
(12) Patent Application Publication
(10) Pub. No.: US 2011/0238116 A1
(43) Pub. Date: Sep. 29, 2011

(54) POLYGONAL ROD

(76) Inventor: Masaki Takemoto, Niigata (JP)

(21) Appl. No.: 12/730,374

(22) Filed: Mar. 24, 2010

(51) Int. Cl. A61B 17/70

(52) U.S. Cl. 606/261

(57) ABSTRACT

To provide a polygonal rod, which is formed into a polygonal shape provided with hollow portions formed in an inside thereof with use of a plurality of T-shaped longitudinal members each having a T-shaped cross-section, and which sufficiently has resilience after bending of the rod. The polygonal rod has a longitudinal-pole-like entire shape and a polygonal outer peripheral shape in a cross-section thereof, the polygonal rod including at least a plurality of T-shaped longitudinal members (10) each having a T-shaped cross-section, to exhibit a hollow shape.

### BACKGROUND OF THE INVENTION

1. Field of the Invention

The present invention relates to a polygonal rod, in particular, a new improvement for providing a structure in which at least three T-shaped longitudinal members each having a T-shaped cross-section are provided to form a polygonal shape provided with at least three hollow portions formed in an inside thereof, the structure sufficiently having resilience even after the rod is bent in any direction.

2. Description of the Related Art

Conventionally-used this type of polygonal rod has been merely manufactured and has not been applied for a patent. Therefore, Patent Document and the like are not disclosed herein. Instead, structures of commercially available polygonal rods can be exemplified in FIGS. 8, 9 and 10.

Specifically, FIG. 8 illustrates a hexagonal hollow rod 1. The hexagonal hollow rod 1 includes six flat-plate-like longitudinal members 2 each formed of a longitudinal flat-plate member, and tapered outer-wall side-surfaces 3 formed on both sides of each of the flat-plate-like longitudinal members 2 are brought into contact with each other and then connected to each other by an adhesive or the like.

A hollow portion 4 having a longitudinal hexagonal shape is formed in a space enclosed by the flat-plate-like longitudinal members 2, and the hexagonal hollow rod 1 is formed into a longitudinal shape and is formed so as to be tapered from a rod base on a front side to a rod apex.

Further, FIG. 9 illustrates a conventional hexagonal solid rod 1A. The hexagonal solid rod 1A includes six trigonal longitudinal members 5 each having a trigonal cross-section, and longitudinal side-surfaces 5a of the trigonal longitudinal members 5 are brought into contact with each other and connected to each other by the adhesive or the like. As a result, the hexagonal solid rod 1A is formed into a hexagonal solid column so that an outer peripheral surface thereof exhibits a hexagonal shape in the cross-section thereof.

Further, another conventional structure illustrated in FIG. 10 is, as described in Non-patent Document 1 described later, a flat hexagonal hollow rod. In the flat hexagonal hollow rod, two T-shaped longitudinal members 10 each having a T-shaped cross-section and four flat-plate-like longitudinal members 2 are assembled together, an outer periphery thereof exhibits a horizontal hexagonal shape, and the T-shaped longitudinal members 10 are arranged so as to be opposed to each other.

Further, inner ends 13 of protruding bars 12 formed integrally with the T-shaped longitudinal members 10 so as to extend therefrom are brought into contact with each other and are connected to each other by the adhesive or the like, to thereby form a pair of hollow portions 4.

[Non-patent Document 1] “Present Bamboo Rod”, Toshosha, Jun. 11, 2007, p. 213

The conventional polygonal rods have the structures as described above, and hence the following problems lie therein.

Specifically, in the conventional structure illustrated in FIG. 8, because the hexagonal rod itself is simply formed into a tubular shape, the hollow portion in the cross-section is flatly crushed when the rod is bent, which results in reduction of resilience. Therefore, it is difficult to continue the use thereof for a long period of time.

Further, in the hexagonal solid rod illustrated in FIG. 9, the hollow portion is not provided in the inside thereof. Therefore, the rod itself has heavy weight, and hence it is difficult to reduce weight, and the rod is not sufficiently pliable.

Further, in the rod having the horizontal hexagonal outer peripheral shape illustrated in FIG. 10, only two T-shaped longitudinal members are used, and hence the protruding bars are arranged straightly in only one direction. Therefore, elasticity of the bar changes depending on bending directions, and hence there is a disadvantage that a direction in which the bar can be used appropriately is limited to one direction. In addition, a process for working each member in order to accurately bring the inner ends of the protruding bars protruding toward a space enclosed by the T-shaped longitudinal members into contact with each other takes a long period of time, which results in a problem of extremely low productivity.

### SUMMARY OF THE INVENTION

A polygonal rod according to the present invention has a longitudinal-pole-like entire shape and a polygonal outer peripheral shape in a cross-section thereof, and has a structure in which at least three T-shaped longitudinal members each having a T-shaped cross-section are provided to form at least three hollow portions. The polygonal rod adopts a structure in which: the T-shaped longitudinal members include three T-shaped longitudinal members; the T-shaped longitudinal members interpose therebetween a flat-plate-like longitudinal member; each of the T-shaped longitudinal members has outer-wall side-surfaces, which are formed on both sides of each outer-wall flat-plate-like wall portion thereof, and are held in contact with flat-plate side-surfaces formed on both sides of the flat-plate-like longitudinal member; each of the T-shaped longitudinal members is provided with a protruding bar, which is provided at a center thereof and protrudes to an inner side of the polygonal rod; and inner ends of the protruding bars are held in contact with each other. Further, the polygonal rod adopts a structure in which: the T-shaped longitudinal members include four T-shaped longitudinal members; each of the T-shaped longitudinal members has outer-wall side-surfaces, which are formed on both sides of each outer-wall flat-plate-like wall portion thereof, and are held in contact with each other; each of the T-shaped longitudinal members is provided with a protruding bar, which is provided at a center thereof and protrudes to an inner side of the polygonal rod; and inner ends of the protruding bars are held in contact with each other. Further, the polygonal rod adopts a structure in which each of the outer-wall side-surfaces includes a tapered surface. Further, the polygonal rod adopts a structure in which: each of the outer-wall side-surfaces includes a tapered surface; and each of the flat-plate side-surfaces includes a tapered surface. Further, the polygonal rod adopts a structure in which each of the inner ends of the T-shaped longitudinal members includes a V-shaped protruding portion.

The polygonal rod according to the present invention can provide the following effects due to the above-mentioned structures.

Specifically, for the polygonal rod having a longitudinal-pole-like entire shape and a polygonal outer peripheral shape in a cross-section thereof, there is adopted the structure in which at least three T-shaped longitudinal members each having a T-shaped cross-section are provided to form at least three hollow portions, and hence the polygonal rod is supported by at least three protruding bars. Therefore, it is possible to provide a rod which, in spite of its hollow shape, can keep high elasticity even when being bent in any direction, is not deformed after bending, and has high durability.

Further, it is possible to provide a hollow rod having a regular hexagonal cross-section by adopting the structure in which: the T-shaped longitudinal members include three T-shaped longitudinal members; the T-shaped longitudinal members interpose therebetween a flat-plate-like longitudinal member; each of the T-shaped longitudinal members has outer-wall side-surfaces, which are formed on both sides of each outer-wall flat-plate-like wall portion thereof, and are held in contact with flat-plate side-surfaces formed on both sides of the flat-plate-like longitudinal member; each of the T-shaped longitudinal members is provided with a protruding bar, which is provided at a center thereof and protrudes to an inner side of the polygonal rod; and inner ends of the protruding bars are held in contact with each other. Further, it is possible to provide a hollow rod having a tetragonal cross-section by adopting the structure in which: the T-shaped longitudinal members include four T-shaped longitudinal members; each of the T-shaped longitudinal members has outer-wall side-surfaces, which are formed on both sides of each outer-wall flat-plate-like wall portion thereof, and are held in contact with each other; each of the T-shaped longitudinal members is provided with a protruding bar, which is provided at a center thereof and protrudes to an inner side of the polygonal rod; and inner ends of the protruding bars are held in contact with each other.

Further, by adopting the structure in which each of the outer-wall side-surfaces includes a tapered surface, the T-shaped longitudinal members can be more easily connected to each other. Further, by adopting the structure in which: each of the outer-wall side-surfaces includes a tapered surface; and each of the flat-plate side-surfaces includes a tapered surface, the T-shaped longitudinal members and the flat-plate-like longitudinal members can be more easily connected to each other. Further, by adopting the structure in which each of the inner ends of the T-shaped longitudinal members includes a V-shaped protruding portion, the inner ends of the protruding bars of the T-shaped longitudinal members can be more easily connected to each other.

### BRIEF DESCRIPTION OF THE DRAWINGS

In the accompanying drawings:

FIG. 1 is a perspective view illustrating a cross-section of a polygonal rod according to the present invention;

FIG. 2 is a sectional view illustrating another embodiment of FIG. 1;

FIG. 3 is a process view illustrating how a component of the polygonal rod of FIG. 1 is worked;

FIG. 4 is a process view illustrating how a bamboo 50 worked in FIG. 3 is worked to be formed into a T-shaped column;

FIG. 5 is a process view illustrating how the T-shaped column of FIG. 4 is worked by an angular device so as to provide a T-shaped longitudinal member 10;

FIG. 6 is a perspective view illustrating another embodiment of a protruding bar of the polygonal rod according to the present invention;

FIG. 7 is a perspective view illustrating still another embodiment of the protruding bar of the polygonal rod according to the present invention;

FIG. 8 is a partial perspective view of a conventional hexagonal hollow rod;

FIG. 9 is a partial perspective view of a conventional hexagonal solid rod; and

FIG. 10 is a partial perspective view of a conventional flat hexagonal hollow rod.

### DETAILED DESCRIPTION OF THE PREFERRED EMBODIMENTS

An object of the present invention is to provide a polygonal rod, which is formed into a regular polygonal shape in which at least three T-shaped longitudinal members each having a T-shaped cross-section are provided and at least three hollow portions are provided in an inside thereof, and which sufficiently has resilience even after the rod is bent in any direction.

### Embodiments

Hereinafter, preferred embodiments of a polygonal rod according to the present invention are described with reference to the drawings.

Note that portions which are the same as those of the conventional example are described while being denoted by the same reference symbols.

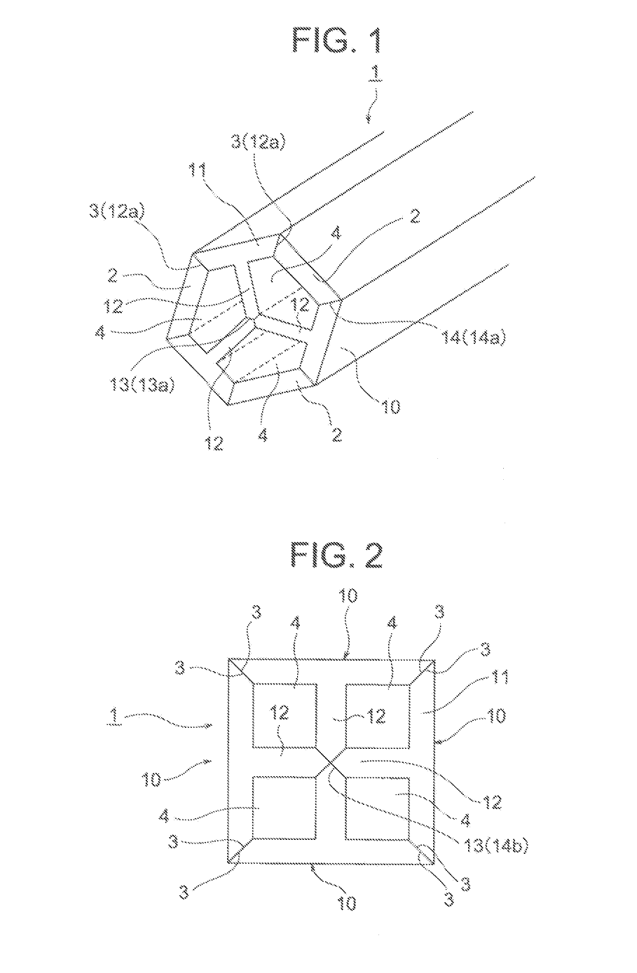

In FIG. 1, the reference numeral 1 denotes a polygonal rod formed of a regular-hexagonal hollow rod. The polygonal rod 1 includes at least three T-shaped longitudinal members 10 each having a T-shaped cross-section and flat-plate-like longitudinal members 2 each connected to both sides of the T-shaped longitudinal member 10.

Each of the T-shaped longitudinal members 10 includes an outer-wall flat-plate-like wall portion 11 and a protruding bar 12, which is provided at the center of the outer-wall flat-plate-like wall portion 11 and protrudes to the inner side of the polygonal rod.

Outer-wall side-surfaces 3 each formed of a tapered surface 12a are formed on the both sides of the outer-wall flat-plate-like wall portion 11 of the T-shaped longitudinal member 10. An inner end 13 of the protruding bar 12 is formed of a V-shaped protruding portion 14b.

On both sides of each of the flat-plate-like longitudinal members 2, flat-plate side-surfaces 14 each formed of a tapered surface 14a are formed. The flat-plate side-surfaces 14 formed on the both sides of each flat-plate-like longitudinal members 2 are connected by an adhesive or the like to the outer-wall side-surfaces 3 formed on the each side of the outer-wall flat-plate-like wall portion 11. In this manner, the polygonal rod 1 formed of the regular-hexagonal hollow rod is formed.

The V-shaped protruding portions 14b are connected to each other by the adhesive or the like, to thereby form three hollow portions 4 in a space enclosed by the T-shaped longitudinal members 10 and the flat-plate-like longitudinal members 2. Therefore, the protruding bars 12 have functions as supporting columns for the hollow portions 4.

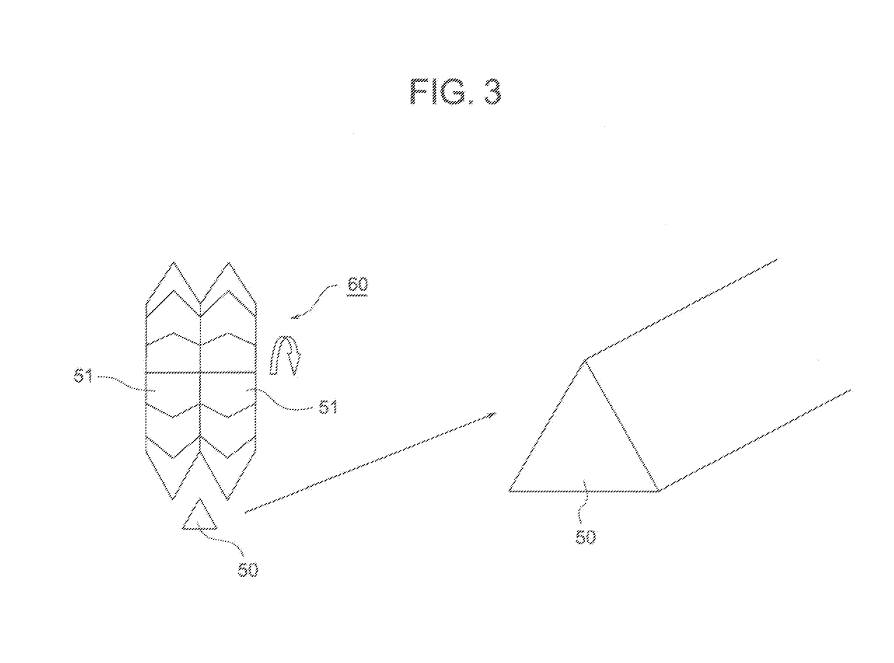

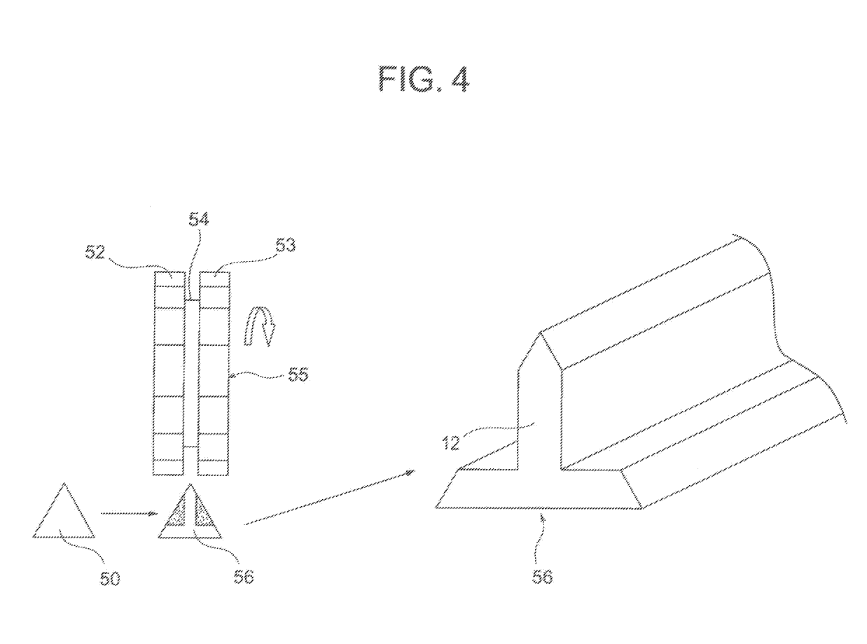

FIG. 2 is an end view according to another embodiment of the present invention, which illustrates the polygonal rod 1 formed of a regular-tetragonal hollow rod.

In FIG. 2, four T-shaped longitudinal members 10 constitute the polygonal rod 1 formed of the regular-tetragonal hollow rod having a regular-tetragonal cross-section by connecting the outer-wall side-surfaces 3 to each other by the adhesive or the like.

The V-shaped protruding portions 14b of the inner ends 13 of the protruding bars 12 of the T-shaped longitudinal members 10 are connected to each other by the adhesive or the like, to thereby form the four hollow portions 4 in the space enclosed by the T-shaped longitudinal members 10.

Next, description is made of the case of manufacturing the T-shaped longitudinal members 10 and the flat-plate-like longitudinal members 2 constituting the above-mentioned polygonal rod 1.

As illustrated in FIG. 3, a bamboo is used as a material, and a longitudinal bamboo 50 is first cut out by an angular cutter device 60 into a rough-trigonal-column shape having an angle of 60°, the angular cutter device 60 having well-known two angular cutters 51 having an angle of 60°. The bamboo 50 is shaved by a side cutter device 55 so that the bamboo 50 having the trigonal-column-like entire shape is formed into a T-shaped column 56 having a T-shaped cross-section, the side cutter device 55 including a pair of well-known side cutters 52 and 53 and a spacer 54 sandwiched therebetween.

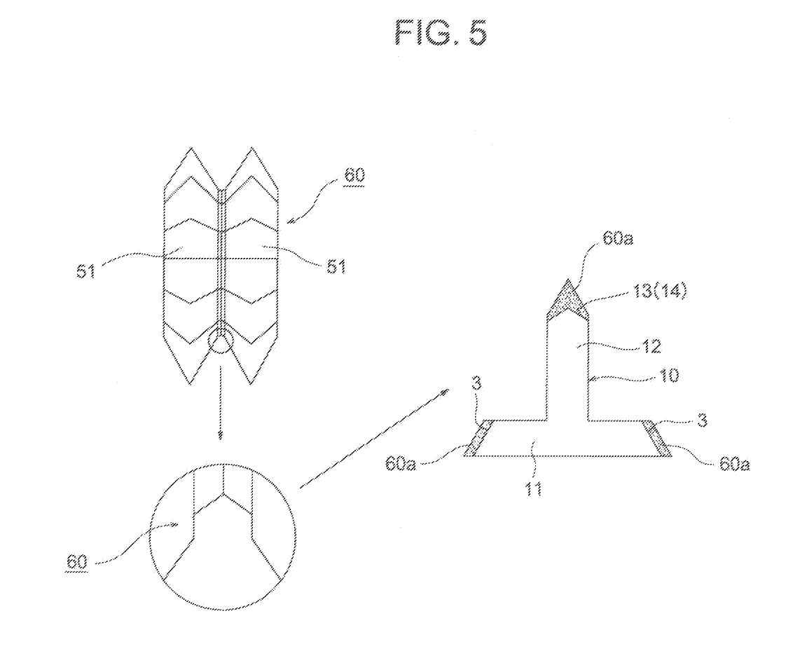

Next, as illustrated in FIG. 5, the above-mentioned T-shaped longitudinal member 10 is formed by shaving the tapered surface 12a and the inner end 13 of the T-shaped column 56 so that angles thereof are changed from 60° to 120° (by shaving dotted portions 60a illustrated in FIG. 15) with use of an angular cutter device in which a shoulder portion having an angle of 60° formed between the angular cutters 51 is cut out so that the angle is changed to 120°, or with use of the angular cutter device 60 including two angular cutters each having an angle of 120°.

That is, use of the pair of angular cutters 51 of 60° having a cutout of 120° enables simultaneous finish workings of the protruding bar 12 and the outer-wall flat-plate-like wall portion 11 of the T-shaped longitudinal member 10.

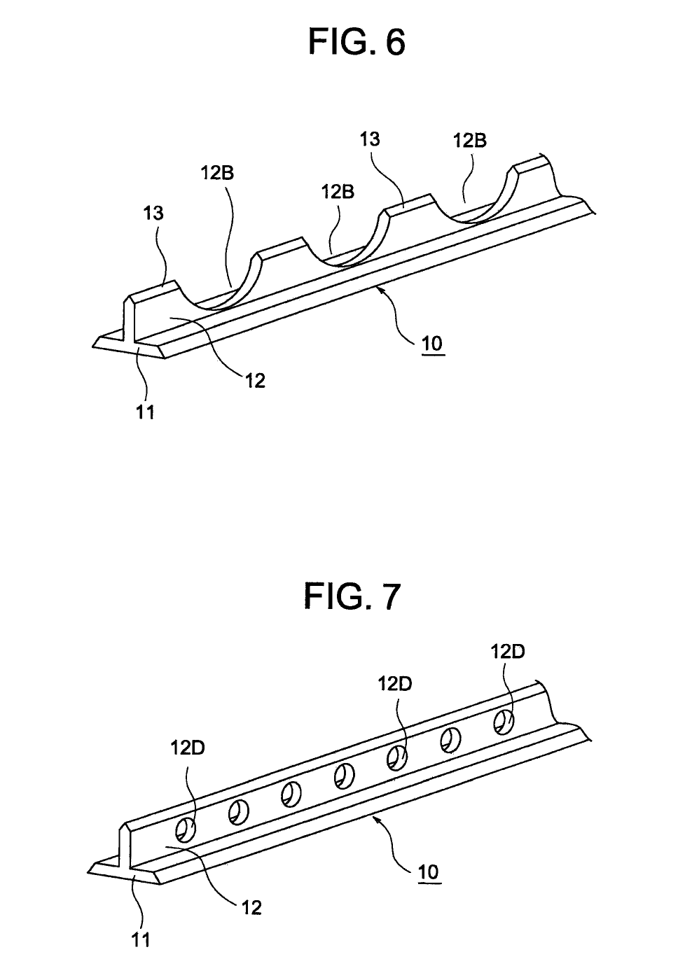

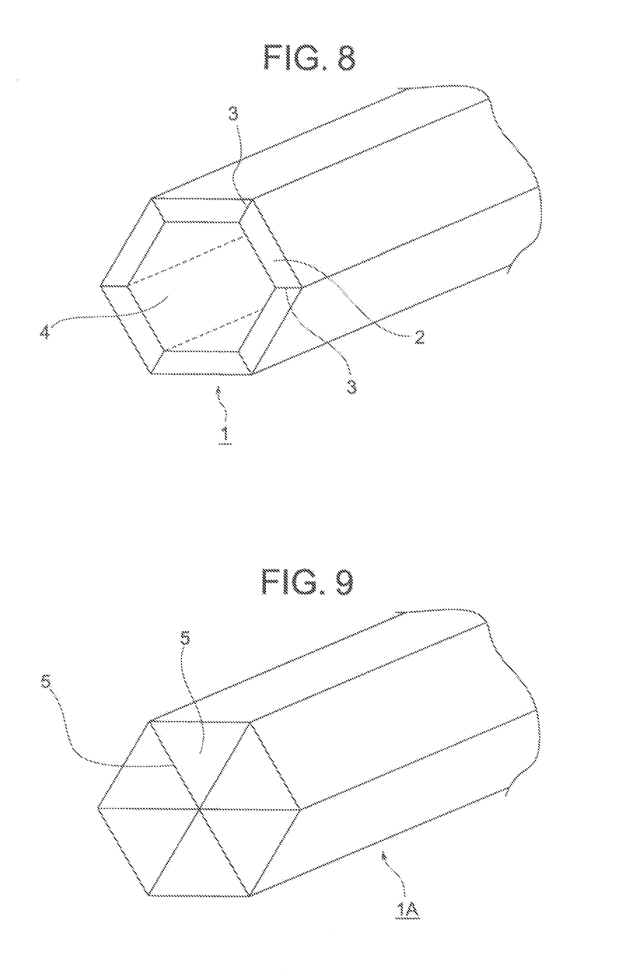

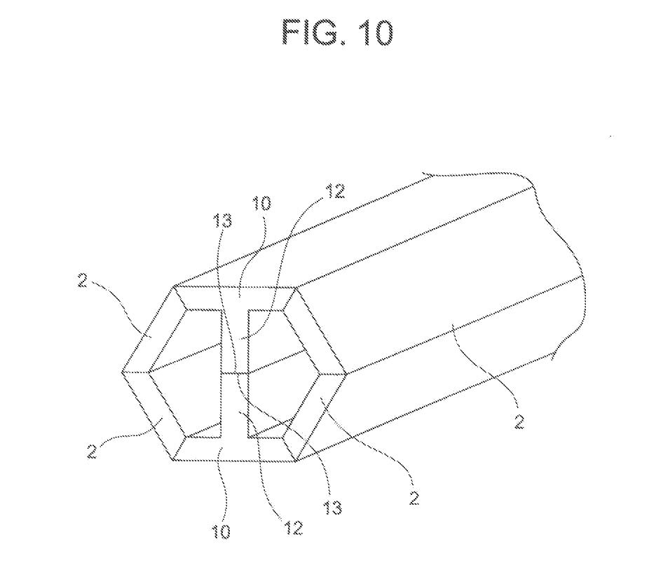

Note that the polygonal rod according to the present invention can be constituted by at least three T-shaped longitudinal members 10. In this case of using the at least three T-shaped longitudinal members 10, when one side or both sides of the outer-wall flat-plate-like wall portions 11 of the T-shaped longitudinal members 10 are extended, it is possible to form the hexagonal hollow rod illustrated in FIG. 1 without using the separate flat-plate-like longitudinal members 2. Further, the protruding bars 12, which are provided at the centers of the outer-wall flat-plate-like wall portions 11 of the T-shaped longitudinal members 10 and protrude to the inner side of the polygonal rod, are generally wall-like structures. As illustrated in FIGS. 6 and 7, by appropriately providing holes 12D or cutouts 12B in the protruding bar 12, it is possible to further reduce weight thereof without detracting the effect of the present invention. Further, in the structures illustrated in FIGS. 1 and 2, the protruding bars 12 are assembled together into a Y-shape (star shape) and a cross shape, respectively. Therefore, sufficient resilience can be provided even after the rod is bent in any direction.

1. A polygonal rod having a longitudinal-pole-like entire shape and a polygonal outer peripheral shape in a cross-section thereof, the polygonal rod comprising at least three T-shaped longitudinal members (10) each having a T-shaped cross-section, to thereby form at least three hollow portions (4).

2. A polygonal rod according to claim 1, wherein:
the T-shaped longitudinal members (10) interpose therebetween a flat-plate-like longitudinal member (2);
each of the T-shaped longitudinal members (10) has outer-wall side-surfaces (3), which are formed on both sides of each outer-wall flat-plate-like wall portion (11) thereof, and are held in contact with flat-plate side-surfaces (14) formed on both sides of the flat-plate-like longitudinal member (2);
each of the T-shaped longitudinal members (10) is provided with a protruding bar (12), which is provided at a center thereof and protrudes to an inner side of the polygonal rod; and
inner ends (13) of the protruding bars (12) are held in contact with each other.

3. A polygonal rod according to claim 1, wherein:
the T-shaped longitudinal members (10) comprise four T-shaped longitudinal members (10);
each of the T-shaped longitudinal members (10) has outer-wall side-surfaces (3), which are formed on both sides of each outer-wall flat-plate-like wall portion (11) thereof, and are held in contact with each other;
each of the T-shaped longitudinal members (10) is provided with a protruding bar (12), which is provided at a center thereof and protrudes to an inner side of the polygonal rod; and
inner ends (13) of the protruding bars (12) are held in contact with each other.

4. A polygonal rod according to claim 2 or 3, wherein each of the outer-wall side-surfaces (3) comprises a tapered surface (12a).

5. A polygonal rod according to claim 2, wherein:
each of the outer-wall side-surfaces (3) comprises a tapered surface (12a); and
each of the flat-plate side-surfaces (14) comprises a tapered surface (14a).

6. A polygonal rod according to any one of claims 1 to 3 and 5, wherein each of the inner ends (13) of the T-shaped longitudinal members (10) comprises a V-shaped protruding portion (14b).

7. A polygonal rod according to claim 4, wherein each of the inner ends (13) of the T-shaped longitudinal members (10) comprises a V-shaped protruding portion (14b).
